(19) United States
(12) Patent Application Publication
(10) Pub. No.: US 2007/0036852 A1
(43) Pub. Date: Feb. 15, 2007

(54) Rapidly dispersing/disintegrating compositions

(76) Inventors: Harsha M. Dabhade, Kolshet Thane (IN); Pankaj Umakant Attarde, Pune (IN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/500,139

(22) Filed: Aug. 7, 2006

(30) Foreign Application Priority Data
Aug. 12, 2005 (GB) 0516604.6

(51) Int. Cl. A61K 31/405; A61K 9/20

(52) U.S. Cl. 424/464; 514/419

(57) ABSTRACT

The present invention relates to a rapidly dispersing/disintegrating, taste masked oral ondansetron dosage forms and a simple and economic process for their manufacture which can be easily scaled up. In particular, the present invention relates to a compressed dosage form for oral administration capable of being rapidly disintegrated comprising a bitter active pharmaceutical ingredient, a pharmaceutically acceptable polymer of methacrylic acid with divinylbenzene, and at least one further excipient, a process for the manufacture of such a compressed dosage form, compressed dosage forms obtainable from such a process and the use of Polacrillin potassium for the purpose of taste masking in a rapidly disintegrating dosage form.

The present invention relates to rapidly dispersing/disintegrating, taste masked oral ondansetron dosage forms and simple and economic processes for their manufacture which can be easily scaled up. In particular, the present invention relates to compressed dosage forms for oral administration capable of being rapidly disintegrated and comprising a bitter active pharmaceutical ingredient, a pharmaceutically acceptable polymer of methacrylic acid with divinylbenzene, and at least one further excipient; a process for the manufacture of such a compressed dosage form; compressed dosage forms obtainable from such processes and the use of Polacrillin potassium for the purpose of taste masking in a rapidly disintegrating dosage form.

Ondansetron is a selective and potent antagonist of 5-hydroxytryptamine (5HT) at 5HT3 receptors. It is known to be useful in the treatment of a variety of conditions where administration of 5HT3 receptor antagonists has been shown to be beneficial, e.g. emesis and anxiety. In several clinical studies ondansetron has been shown to be effective for the treatment of emesis, in particular nausea and vomiting associated with cancer chemotherapy and radiotherapy and post-operatively occurring nausea.

While in general oral administration in the form of a conventional tablet, pill or capsule constitutes a preferred route for administration of pharmaceuticals, this does not hold true in the case of treatment of pediatric or geriatric patients. These groups of patients sometimes have difficulties in swallowing such compositions. Therefore, dosage forms that can disintegrate readily in the mouth are a useful alternative for these groups of patients.

However, it is known that ondansetron hydrochloride dihydrate tastes very bitter, and this is, of course, a very disadvantageous property for its use in solid dosage forms that disintegrate rapidly in the mouth.

EP 793495 discloses a freeze-dried dosage form comprising ondansetron in the form of its free base that disintegrates rapidly in the mouth.

However, the process step of freeze-drying is cumbersome and lengthy and creates a challenge for up-scaling of the overall production process. Moreover, freeze-dried compositions are not stable enough to be easily handled by the modem high-speed packaging machines. There is thus a need for a simpler production process which provides an easy-to-handle Ondansetron formulation.

Polacrillin Potassium is the potassium salt of a unifunctional low-cross-linked carboxylic cation-exchange resin prepared from methacrylic acid and divinylbenzene (CAS Nr. 39394-76-5). It is listed in the European Pharmacopoeia 5.0, the United States Pharmacopoeia 28-NF 23 and also listed in FDA's inactive ingredient guide.

Polacrillin potassium which is also known as Amberlite resin IRP88 (obtainable from Rohm and Haas Company), can act as a tablet and capsule disintegrant. Use of Polacrillin potassium as disintegrant in manufacturing of fast disintegrating tablets has been described (see, for example, lines 12-15 on page 6 of PCT application WO99/18965).

This disintegration action of Amberlite resin IRP88 is owing to its extremely large swelling capacity and fairly good rate of water absorption upon hydration as compared to other disintegrants like corn starch, alginic acid and Sodium CMC.

US 2005/0036977 discloses a taste masked resinate that contains a water-insoluble active substance complexed to an ion-exchange resin; among them Polacrillin is mentioned. In a first step, the complex of ion-exchange resin and the water-insoluble bitter active pharmaceutical substance is formed in an aqueous solution. Subsequently, freeze-drying is employed to remove water.

There is thus a need for an easier-to-handle dosage form where the disintegrant at the same time masks the taste of bitter ingredients, like bitter active pharmaceutical ingredients.

The present invention solves the problems of the prior art by incorporating Polacrillin into compressed compositions of Ondansetron. It has surprisingly been found that Polacrillin can serve the two functions of assuring to rapidly disperse the drug from the compressed dosage form and of masking the taste of such dispersed bitter drugs to make the formulation patient compliant at the same time.

The present work relates to rapidly dispersing/disintegrating oral dosage forms, in particular tablets, of bitter active pharmaceutical ingredients, in particular of Ondansetron, and to methods of producing such oral dosage forms. The oral dosage forms disclosed by the present invention can be manufactured using conventional manufacturing equipment. It is thus possible to produce them with a beneficial cost structure.

In particular, the present invention relates to a compressed dosage form for oral administration which is capable of being rapidly disintegrated, for example when put into the oral cavity or into a glass of water, comprising a bitter active pharmaceutical ingredient (API); a pharmaceutically acceptable polymer of methacrylic acid with divinylbenzene, which is preferably a low crosslinked carboxylic cation exchange resin, and which most preferably is the potassium salt thereof which is commonly known under the name Polacrillin potassium (CAS 39394-76-5); and at least one further excipient.

Capable of being rapidly disintegrated within the meaning of the present invention means that the compressed dosage form of the present invention shows a disintegration time (according to item 2.9.1 of the Eur. Pharm.) of less than 1 min.

A bitter API within the meaning of the present invention has a bitterness value (cf. European Pharmacopoeia 5.0 (Eur. Pharm.) item 2.8.15) of more than 100, preferably more than 200, more preferably more than 500 and even more preferably more than 2000.

In particular, the bitter API is a bitter anti-emetic, like flunarizin, beathistin, dimenhydrinat, meclozin, diphenhydramin, an antagonist of 5-hydroxytryptamine at 5HT3 receptors or alizaprid, but preferably is an antagonist of 5-hydroxytryptamine at 5HT3 receptors. Such serotonin receptor antagonists are, for example, dolasetronmesilat, granisetron, tropisetron and ondansetron with ondansetron being most preferred.

Ondansetron contains one chiral centre and thus exists in the form of optical isomers (i.e. enantiomers). The present invention relates to all isomers of ondansetron and its pharmaceutically acceptable salts. Also all tautomeric and optical forms, as well as mixtures thereof, also racemic mixtures, are included. Preferred forms of Ondansetron are its hydrochloride and its free base.

In a preferred embodiment, the bitter API, and in particular Ondansetron, is present in an amount of from 1% to 10% w/w, more preferably from 2% to 8% w/w and most preferably from 3% to 7% w/w.

In a preferred embodiment, the amount of Ondansetron in the dosage form of the present invention, expressed as the amount of its free base, is from 0.5 to 40 mg, more preferably from 1 to 20 mg, even more preferably from 3 to 10 mg.

In a further preferred embodiment the pharmaceutically acceptable polymer of methacrylic acid with divinylbenzene, preferably the above mentioned low crosslinked carboxylic cation exchange resin made therefore, and most preferably Polacrillin potassium, is present in an amount of from 0.1 to 8% w/w, preferably from 2% to 6% and more preferably from 3 to 5% w/w.

Suitable excipients for use in the present invention are preservatives, fillers, sweeteners, flavoring agents, coloring agents, disintegrating agents, glidants, antiadherent, lubricants and mixtures thereof.

In a preferred embodiment the further excipient is a further disintegrant, in particular an insoluble or poorly soluble cross-linked polymeric disintegrant, preferably present in an amount of from 0.1 to 5% w/w. Such further disintegrating agents are preferably selected from sodium starch glycolate, crospovidone, croscarmellose sodium and low substituted hydroxy propyl cellulose, with sodium starch glycolate being particularly preferred. It is preferably added to a final concentration of below 5%, more preferably below 3%. The overall amount of disintegrating agent, that is the amount of pharmaceutically acceptable polymer of methacrylic acid with divinylbenzene and of the further disintegrant together is preferably around 5.5%.

In a further preferred embodiment the further excipient is filler present in an amount of from 15% to 90%. Particularly suitable fillers are lactose, mannitol, sorbitol, xylitol, powdered cellulose, microcrystalline cellulose, calcium carbonate, dibasic calcium phosphate and mixtures thereof. In a preferred embodiment the filler is a cellulose-type filler. More preferably it is selected from powdered cellulose and microcrystalline cellulose. Cellulose type filler can be used alone or in combination with (a) further soluble filler(s). The compressed oral dosage forms of the present invention most preferably contain microcrystalline cellulose as filler used alone or in combination with any one of the above-mentioned fillers. In a further preferred embodiment filler is a mixture of microcrystalline cellulose and mannitol. Although other fillers such as calcium carbonate, dibasic calcium phosphate etc can be added, microcrystalline cellulose is most preferred. Microcrystalline cellulose in the range of from 15% to 90% is found suitable. It is preferred that, in those embodiments where microcrystalline cellulose is not used as the sole filler, the microcrystalline cellulose is present in an amount of from 15% to 99% of total filler, preferably of from 20% to 90% of total filler, more preferably of from 25% to 85% of total filler.

Microcrystalline cellulose has good compressibility characteristics and tablets produced thereof are of sufficient hardness and along with Polacrillin potassium, rapidly disintegrating tablets of sufficient hardness are successfully produced which in mouth disintegrate within 60 seconds and more preferably within 30 seconds. The combination of microcrystalline cellulose and Polacrillin potassium is most suitable for the present work.

Amongst soluble fillers are lactose, sorbitol, xylitol and mannitol. Mannitol by virtue of its negative heat of dissolution, pleasant taste and mouth feel is good filler for mouth dissolve tablets.

In a further preferred embodiment the excipient is a glidant. In particular it is a glidant selected from colloidal silicon dioxide and talc, preferably colloidal silicone dioxide and/or talc is present in an amount of from 0.1% to 3.0%.

In a further preferred embodiment the excipient is a lubricant. Particularly suitable lubricants are calcium stearate, magnesium stearate, sodium stearyl fumarate, stearic acid in particular stearic acid and/or its salts.

It is preferred that the lubricant be present in an amount from 0.5% to 5.0%.

Faculatatively, talc, colloidal silicon dioxide, magnesium or calcium stearate can be added to improve flow properties of the blend.

In a further preferred embodiment the excipient is a sweetener. Particularly suitable sweeteners are powdered sugar, saccharine, aspartame, cyclamates, in particular selected from the group consisting of cyclamate or salts thereof; saccharin or salts thereof; and aspartame. Aspartame in concentrations of 4-12%, preferably in concentrations of 6 - 10% can be added.

In a further preferred embodiment the excipient is a flavouring agent. Particularly suitable flavouring agents are vanilla, banana, strawberry, caramel, orange, lemon and cherry, with strawberry being particularly preferred.

A total amount of sweetner and flavouring agent in the range of 5 - 15% is found most suitable.

As explained above, the oral dosage forms are compressed, yet they are still capable of disintegrating rapidly. Thus, in a further preferred embodiment the density of the compressed dosage form is from 0.5 g/cm³ to 2.0 g/cm³, preferably from 0.8 to 1.5 g/cm³

In preferred embodiment the compressed dosage forms of the present invention show a disintegration time from 5s to 50s, more preferably from 5s to 40s, most preferably from 5s to 30s.

The compressed dosage forms of the invention have an improved crushing strength when compared to freeze-dried formulations of the prior art. Thus, the present invention, in a preferred embodiment, relates to compressed dosage forms having a crushing strength (according to item 2.9.8 of the European Pharmacopoeia 5.0) of from 10 to 50 N, preferably of from 12 to 40 N, more preferably from 15 to 30 N.

The compressed dosage forms of the present invention are less friable compared to freeze dried formulations of the prior art. Thus, the present invention in a preferred embodiment relates to compressed dosage forms having a friability (according to item 2.9.7 of the European Pharmacopoeia 5.0) of less than 1%, preferably less than 0.9% and most preferably less than 0.7%.

The compressed oral dosage forms of the present invention do not require binder in the formulation. They can be formulated either by simple blending followed by direct compression or by wet granulation technique. The tablets produced by each technique have very good tensile strength and sufficient hardness to make it suitable to pack on high speed packing machines without requiring any special care and precaution for handling as well as packing of the tablets. The tablets maintain the sufficient hardness throughout their shelf life. This is a great advantage of the present formulation over freeze dried tablet formulations which lack hardness and require special care in handling.

The present invention also relates to a process for the manufacture of a compressed dosage form for oral administration of a bitter active pharmaceutical ingredient capable of being rapidly disintegrated, comprising the steps of

a) Mixing the bitter active pharmaceutical ingredient, a pharmaceutically acceptable polymer of methacrylic acid with divinylbenzene which is preferably a low crosslinked carboxylic cation exchange resin, and most preferably Polacrillin potassium, and at least one further excipient; and

b) compressing the mixture obtained from step a) into a compressed dosage form.

As an additional step, a lubricant can be added to the mixture obtained from step a) and further mixing before compressing the mixture into a compressed dosage form.

If water is added during the formulation step, then—in one embodiment of the invention—it is used in an amount that does not lead to complete swelling of the pharmaceutically acceptable polymer of methacrylic acid with divinylbenzene, in particular the Polacrillin potassium, so that it is assured that optimal disintegrating properties are retained. The amount of water used during the process of the invention can be adjusted so as to retain optimal functionality of Polacrillin as a disintegrant.

Alternatively, the present invention relates to a process for the manufacture of a compressed dosage form for oral administration of a bitter active pharmaceutical ingredient capable of being rapidly disintegrated, comprising the steps of

a) Dispersing the polymer of methacrylic acid with divinylbenzene, which is preferably a low crosslinked carboxylic cation exchange resin, and most preferably Polacrillin potassium, and the bitter active pharmaceutical ingredient in water;

b) Mixing the inactive excipients of the granular portion of the dosage form in a blender;

c) Granulating the inactives of step b) with the dispersion of step a);

d) drying of the so obtained wet mass to form granules;

e) Mixing the granules so obtained with extragranular inactives; and

e) Compressing the granules obtained from step e) into a compressed dosage form.

As an additional step, a lubricant can be added to the mixture obtained from step e) and further mixing can be applied before compressing the mixture into a compressed dosage form.

In a preferred embodiment, however, the process for the manufacture of a compressed dosage of the invention is carried out essentially in the absence of aqueous solutions. This can be done by intensely mixing and blending the ingredients in the powder form, as exemplified in example 3.

The compression force required to carry out compression usually varies with the compression machine. For the present invention, the compression force is found to vary from 20 kN -90 kN and more preferably around 50 kN.

In a preferred embodiment, the bitter active pharmaceutical ingredient used in the process is an anti-emetic, in particular an antagonist of 5-hydroxytryptamine at 5HT3 receptors, more particularly ondansetron, in particular ondansetron hydrochloride or ondansetron free base.

The present invention also relates to the use of a pharmaceutically acceptable polymer of methacrylic acid with divinylbenzene, preferably a low crosslinked carboxylic cation exchange resin made there from, and most preferably the use of Polacrillin potassium, for the dual purpose of a disintegrant and a taste masking agent. Polacrillin potassium can therefore be used as a disintegrant and for the purpose of taste masking. In a preferred embodiment, polacrillin potassium at the same time also acts as a binder. It is preferred that the dosage form is not a freeze-dried dosage form. Thus, there is the additional advantage that this use of Polacrillin potassium allows the concentration of the further taste masking agents to be lowered.

In one embodiment of the invention contact of polacrillin potassium with excessive amounts of aqueous solutions during the formulation steps is avoided. This can be achieved, for example, by blending polacrillin potassium, the bitter API and the further ingredients of the tablet in the dry state and then compressing the resulting blend to obtain a tablet.

The compressed oral dosage forms of the present invention wherein the API is a 5HT competitor like ondansetron can be used for the treatments of conditions where competitors of 5HT at 5HT3 receptors have shown to be therapeutically useful. These conditions include the conditions listed on page 6 of WO96/15785, with a particular emphasis given to the treatment of emesis regardless of its cause. Emesis as used herein includes all forms emesis as listed on pages 6 and 7 of WO96/15785.

### EXAMPLES

The following examples describe dosage forms of the present invention in detail, but they are not to be construed to be in any way limiting for the present invention.

### Example 1

| Ingredients | Milligram per tablet |
|---|---|
| Ondansetron hydrochloride dihydrate | 10.00 |
| Lactose | 50.00 |
| Microcrystalline cellulose | 27.00 |
| Pregelatinised starch | 6.00 |
| Sodium starch glycolate | 22.00 |
| Aspartame | 7.50 |
| Magnesium Stearate | 0.50 |
| Orange flavour | 2.00 |
| Total | 125.00 |

Manufacturing Process

1 Mix ondansetron hydrochloride dihydrate, Lactose, Microcrystalline cellulose, pregelatinised starch, sodium starch glycolate, aspartame, flavour together

2 Add magnesium stearate and mix further.

3 Compress the blend into tablets

Observation—

Taste—highly bitter taste.

Dispersion time—About 1 minute

### Example 2

| Ingredients | Milligram per tablet |
|---|---|
| Ondansetron hydrochloride dihydrate | 10.00 |
| Microcrystalline cellulose | 210 |
| Polacrillin potassium | 10.00 |
| Iron oxide yellow | 3.00 |
| Aspartame | 12.00 |
| Magnesium stearate | 2.50 |
| Purified talc | 2.00 |
| Colloidal silicon dioxide | 2.50 |
| Sodium starch glycolate | 5.00 |
| Orange flavour | 6.00 |
| Total | 263 |

Manufacturing Process

1 Disperse Ondansetron HCL and Polacrillin potassium in water.

2 Granulate the microcrystalline cellulose with dispersion from step 1.

3 Dry the wet mass in tray drier to produce granules

4 Mix the dried granules from above step with magnesium stearate, aspartame, talc, colloidal silicon dioxide, sodium starch glycolate Iron oxide yellow and orange flavor.

5 Compress the lubricated granules from above step into tablets

Observation—

Taste—Excellent taste

Dispersion time—10-15 seconds

### Example 3

| Ingredients | Milligram per tablet |
|---|---|
| Ondansetron Base | 8.00 |
| Microcrystalline cellulose | 30.00 |
| Polacrillin potassium | 6.00 |
| Mannitol | 70.00 |
| Aspartame | 12.00 |
| Calcium Stearate | 6.00 |
| Purified talc | 1.00 |
| Colloidal silicon dioxide | 1.00 |
| Sodium starch glycolate | 2.00 |
| Flavoring agent | 4.00 |
| Total | 140.00 |

Manufacturing Process

1 Mix Ondansetron base, Microcrystalline cellulose, Polacrillin potassium, mannitol, aspartame, talc, colloidal silicon dioxide, sodium starch glycolate and strawberry flavour together

2 Add calcium stearate and blend

3 Compress the lubricated blend into tablets

Observation—

Taste—Excellent taste

Dispersion time—10-15 seconds

Discussion: In Example 1, no Polacrillin potassium is present and the taste is bitter, moreover, even though a high level of disintegrant(17%) is added, the disintegration time is still around 1 minute.

In the wet granulation process of Example 2 polacrilin potassium is present. Taste is excellent and the level of further disintegrating agents required to achieve a desired disintegration time is very low.

In the direct compression process of Example 3 dosage forms are obtained with desired taste, a desired short disintegration time, while overall using a simple process which is easy to be scaled up.

Conclusion: The rapidly dispersible compressed dosage forms according to the present formulation are rapidly disintegratable and show the following desirable properties:

They have substantially good organoleptic properties

There is no need for special precautions for packaging and handling of these compositions

Their production does not require special environmental conditions, such as reduced temperature, low relative humidity and/or pressure.

Their manufacture does not involve use of organic solvents. Thus, these compositions are producable in an environmentally friendly manner.

They can be produced by employing simple equipment and the production process can be scaled up easily.

1. Compressed dosage form for oral administration capable of being rapidly disintegrated comprising a bitter active pharmaceutical ingredient, a pharmaceutically acceptable polymer of methacrylic acid with divinylbenzene, and at least one further excipient.

2. Compressed dosage form of claim 1, wherein the pharmaceutically acceptable polymer of methacrylic acid with divinylbenzene is polacrilin potassium.

3. Compressed dosage form of claim 1, wherein the bitter active pharmaceutical ingredient is an antagonist of 5-hydroxytryptamine at 5HT3 receptors.

4. Compressed dosage form of claim 1, wherein the active pharmaceutical ingredient is present in an amount of from 1% to 10% w/w.

5. Compressed dosage form of claim 2, wherein Polacrilin potassium is present in an amount of from 1 to 8 % w/w.

6. Compressed dosage form of claim 1, wherein the further excipient is a disintegrant.

7. Compressed dosage form of claim 1, wherein the further excipient is a filler.

8. Compressed dosage form of claim 1, wherein the excipient is a glidant.

9. Compressed dosage form of claim 1, wherein the excipient is a lubricant.

10. Compressed dosage form of claim 9, wherein the lubricant is present in an amount from 0.5 to 5.%.

11. Compressed dosage form of claim 1, wherein the excipient is a sweetener.

12. Compressed dosage form of claim 11, wherein the sweetener is present in an amount of from 4 to 10.0%

13. Compressed dosage form of claim 1 wherein compressed dosage form has a crushing strength of from 10 to 50 N.

14. Compressed dosage form of claim 13, wherein the disintegration time is less than 1 min.

15. Process for the manufacture of a compressed dosage form for oral administration of a bitter active pharmaceutical ingredient capable of being rapidly disintegrated according to claim 1 claims, comprising a direct compression process or a wet granulation process.

16. Process of claim 15 comprising the steps of
a. Mixing the bitter active pharmaceutical ingredient, the pharmaceutically acceptable polymer of methacrylic acid with divinylbenzene, which is in particular polacrilin potassium, and at least one further excipient;
b. Compressing the mixture obtained from step a) into a compressed dosage form, wherein optionally a lubricant can be added to the mixture obtained from step a and the resulting mixture is then further mixed before compressing the mixture into a compressed dosage in step b).

17. Process of claim 15 comprising the steps of
a) Dispersing the polymer of methacrylic acid with divinylbenzene and the bitter active pharmaceutical ingredient in water;
b) mixing inactives in a blender;
c) Granulating the inactives obtained from step b) with the dispersion of step a);
d) drying the wet mixture obtained from step d) to form granules;
e) Mixing the granules obtained from step d) with extragranular inactives; and
f) compressing the granules obtained from step e) into a compressed dosage form.

18-19. (canceled)

20. Compressed dosage form of claim 1 wherein the antagonist of 5-hydroxytryptamine at 5HT3 receptors is ondansetron, or a pharmaceutically acceptable salt, solvate, enantiomer or mixtures thereof including a racemic mixture.

21. Compressed dosage form of claim 13, wherein the disintegration time is from 5s to 30s.
